(19) United States
(12) Patent Application Publication
(10) Pub. No.: US 2025/0286385 A1
(43) Pub. Date: Sep. 11, 2025

(54) SEA-LAND COLLABORATION-BASED MULTI-ENERGY COUPLING LOW-CARBON NEW ENERGY SYSTEM AND OPTIMAL SCHEDULING METHOD

(71) Applicants: Zhejiang University, Hangzhou (CN); Zhejiang Baimahu Laboratory Co., Ltd., Hangzhou (CN); Jiaxing Research Institute, Zhejiang University, Jiaxing (CN)

(72) Inventors: Xiang GAO, Hangzhou (CN); Chenghang ZHENG, Hangzhou (CN); Can ZHOU, Jiaxing (CN); Yuhao SHAO, Hangzhou (CN); Haidong FAN, Hangzhou (CN); Chao YANG, Hangzhou (CN); Yifan WANG, Hangzhou (CN); Yongxin ZHANG, Jiaxing (CN); Weiming TENG, Hangzhou (CN); Zhongyang ZHAO, Hangzhou (CN); Weihong WU, Hangzhou (CN); Weiguo WENG, Jiaxing (CN)

(73) Assignees: Zhejiang University, Hangzhou (CN); Zhejiang Baimahu Laboratory Co., Ltd., Hangzhou (CN); Jiaxing Research Institute, Zhejiang University, Jiaxing (CN)

(21) Appl. No.: 19/219,938

(22) Filed: May 27, 2025

(30) Foreign Application Priority Data
May 28, 2024 (CN) 202410673237.X

(51) Int. Cl. H02J 3/46; C07C 1/12; C07C 29/152; C25B 1/04; C25B 1/27; C25B 11/052; C25B 11/085; C25B 11/089; H02J 3/28

(52) U.S. Cl. CPC H02J 3/466; C07C 1/12; C07C 29/152; C25B 1/04; C25B 1/27; C25B 11/052; C25B 11/085; C25B 11/089; H02J 3/28; H02J 2300/10; H02J 2300/24; H02J 2300/28; H02J 2300/40

(57) ABSTRACT

A sea-land collaboration-based multi-energy coupling low-carbon new energy system includes a low-carbon power generation unit, a green fuel synthesis unit and an energy storage device which are arranged on a sea and an island, a green fuel comprehensive utilization unit and a carbon capture device which are arranged on the island and/or on land, and a multi-energy flow coupling-based sea-land collaborative low-carbon intelligent control center. The system generates power using abundant and stable solar energy and wind energy on the sea and the island, prepares hydrogen and ammonia using seawater, and the green fuel synthesis unit prepares green fuels using the prepared hydrogen and carbon dioxide produced by the system, such that the use of coal and natural gas in the green fuel comprehensive utilization unit is reduced; meanwhile, produced carbon dioxide is used as raw materials to prepare green fuels again.

### CROSS-REFERENCE TO RELATED APPLICATIONS

This application claims priority to Chinese Patent Application No. 202410673237.X with a filing date of May 28, 2024. The content of the aforementioned application, including any intervening amendments thereto, is incorporated herein by reference.

### BACKGROUND OF THE INVENTION

### 1. Technical Field

The disclosure belongs to the technical field of low-carbon integrated energy power generation, and particularly relates to a sea-land collaboration-based multi-energy coupling low-carbon new energy system and an optimal scheduling method.

### 2. Description of Related Art

Under the background of “carbon peaking and carbon neutrality”, energy systems are gradually transformed from traditional fossil energy systems to low-carbon and diversified new energy systems. In the transformation process, the vigorous development of clean and low-carbon coupled energy systems including new energy becomes an important orientation of new energy system construction. However, the power output of new energy power generation is intermittent, random and volatile, which makes it an inevitable choice for the construction of low-carbon new energy systems to vigorously promote the construction of multi-energy complementary integrated energy supply systems. In the construction process of low-carbon new energy systems, carbon reduction heavily relies on industry transformation and upgrading and the increase in the proportion of external renewable energy due to the deficiency of onshore energy resources in coastal regions in China. Restrained by the economic development and energy and power structures in western regions, the supply of renewable energy to the coastal regions from the western regions to support the “carbon peaking and carbon neutrality” strategy has reached a bottleneck, which makes it necessary to deeply exploit and use abundant offshore energy resources in the coastal regions to construct sea-land collaborative new energy systems.

However, how to construct a sea-land collaborative new energy system and realize optimal scheduling of resources thereof faces a plurality of challenges: the time granularity is coarse, the consideration of the volatility of renewable energy resources and constraints of the dynamic flexibility of power supply units is not deep enough, the non-uniformity of renewable energy resource endowments at different levels from sea, land and stations to regions in space and the multi-scale volatility and intermittency in the time dimension need to be comprehensively considered; existing multi-energy complementary coupling is based on performance and efficiency parameters of existing technologies and fails to into full consideration transverse and longitudinal complementation and collaboration of energy systems, and the interaction between technical evaluation indicators such as carbon reduction, economy, safety and flexibility is not clear enough; and most grid-oriented control demands ignore the conversion and utilization process of carbon dioxide and fail to fully tap the flexibility potential of energy production-product production coordinative control.

In view of the above challenges, considering ocean and land energy endowments and the load trend, the construction of an integrated energy production unit with intelligent combination and coupling characteristics for multi-energy integration, diversified energy storage and multi-energy conversion of an island type multi-energy flow coupling system in different scenarios, and an optimal scheduling method is studied, the advantages of coal power and renewable energy are further exploited based on the carbon-controllable migration idea of energy systems, conversion of green fuels is taken into full account, an integrated energy production unit integration scheme for flexible energy supply to the island type multi-energy flow coupling system is proposed, and a multi-energy coupling low-carbon energy system based on coordinated planning, coordinated management, interactive response and mutual complementation and aid of cold-heat-electricity-gas multi-energy flows is constructed to effectively restrain energy volatilities, reduce CO₂ emission and improve system safety and flexibility.

### BRIEF SUMMARY OF THE DISCLOSURE

The objective of the disclosure is to provide a sea-land collaboration-based multi-energy coupling low-carbon new energy system and an optimal scheduling method to solve the problem that there is no low-carbon integrated power supply system based on sea-land collaboration and realize green fuel preparation driven by offshore renewable energy, large-scale storage and transportation of offshore green fuels, efficient, low-carbon, intelligent and diversified comprehensive utilization of green fuels, and more economical, low-carbon and reliable energy supply.

The disclosure relates to a sea-land collaboration-based multi-energy coupling low-carbon new energy system, including a low-carbon power generation unit, a green fuel synthesis unit and an energy storage device which are arranged on a sea and an island, a green fuel comprehensive utilization unit and a carbon capture device which are arranged on the island and/or on land, and a multi-energy flow coupling-based sea-land collaborative low-carbon intelligent control center arranged on the island or on land;
the low-carbon power generation unit generates zero-carbon and/or low-carbon power based on wind energy, light energy and nuclear energy on the sea and/or the island;
the green fuel synthesis unit produces hydrogen and ammonia using the zero-carbon and/or low-carbon power generated by the low-carbon power generation unit and produces methane and methanol using the hydrogen and carbon dioxide from the carbon capture device, and the ammonia, the methane and the methanol are used by the green fuel comprehensive utilization unit as green fuels;
the energy storage device performs charging using surplus power in a case where power is still surplus after the low-carbon power generation unit satisfies a power load, and compensates for a power shortage in a case where the low-carbon power generation fails to satisfy the power load;
the green fuel comprehensive utilization unit generates power preferentially using the green fuels in a case where the power load is still not satisfied after the energy storage device compensates for the power shortage, and generates power by combusting natural gas and/or coal in a case where the power load is still not satisfied after the green fuel comprehensive utilization unit generates power using the green fuels;
the carbon capture device is configured for capturing carbon dioxide produced by the green fuel comprehensive utilization unit and transporting the carbon dioxide into the green fuel synthesis unit;
the sea-land collaborative low-carbon intelligent control center calculates a zero-carbon and/or low-carbon power generation capacity according to acquired parameters of the wind energy, light energy and nuclear energy on the sea and/or the island, and controls operation strategies of the green fuel synthesis unit and the green fuel comprehensive utilization unit according to a power output of the energy storage device and the power load.

In the disclosure, “wind power generation” refers to converting kinetic energy of winds into mechanical kinetic energy and then converting the mechanical energy into electric kinetic energy, that is, a wind turbine rotates under the action of wind power to convert kinetic energy of winds into mechanical energy of a wind turbine shaft, and a generator is driven by the wind turbine shaft to rotate to generate power.

In the disclosure, “light energy power generation” is photovoltaic power generation, which is a technique that directly converts light energy into electric energy using a volta effect produced on a semiconductor interface and is mainly based on a solar cell panel assembly and a controller. Solar cells are connected in series and then packaged and protected to form a large-area solar call panel assembly, which works together with the controller and other components to form a photovoltaic power generation device.

In the disclosure, “nuclear energy power generation” refers to power generation using heat energy released by nuclear fusion of a nuclear reactor. It replaces a boiler configured for thermal power generation with a nuclear reactor and a steam generator, heat is transferred to water in a steam generator, and then steam is formed to push a turbo generator.

Preferably, the low-carbon power generation unit includes one or a combination of more than one of photovoltaic power generation, wind power generation and nuclear power generation.

Preferably, the green fuel synthesis unit includes:
a seawater-electrolysis hydrogen production device driven by a wind-light volatile power supply, configured for producing hydrogen by seawater electrolysis;
an electro-catalytic ammonia production device, configured for synthesizing ammonia by hydrogen and nitrogen;
a methane synthesis device, configured for synthesizing methane using hydrogen and carbon dioxide; and
a methanol synthesis device, configured for synthesizing methanol using hydrogen and carbon dioxide.

A seawater-electrolysis hydrogen production catalyst of a multi-stage array structure and a photovoltaic/wind power volatile renewable energy-based hydrogen production control system are arranged in the seawater-electrolysis hydrogen production device.

Preferably, a surface of an electrode used by the seawater-electrolysis hydrogen production device is covered with an alloy catalyst modified by mixing multiple elements, doped precious metal elements include one or more of nickel, ruthenium, cadmium, molybdenum and platinum, a doped metal loading capacity of an electrode catalyst is less than 0.5 mg/cm², a cathodic overpotential is less than or equal to 300 mV@1000 mA/cm², and an anodic overpotential is less than or equal to 600 mV@1000 mA/cm². An electro-catalytic material in the seawater-electrolysis hydrogen production device is a metal organic complex, a cathodic material is an Al—N₂ or Zr—N₂ cathodic material, and an electrolyte solution is one or more of ionic liquid, ionic liquid/organic solvent, and ionic liquid/organic solvent/water.

The photovoltaic/wind power volatile renewable energy-based hydrogen production control system analyzes and optimizes reaction performance of the seawater-electrolysis hydrogen production device using an electrochemical-heat-fluid coupling numerical model of an electrolytic bath and adjusts in real time operation control of the seawater-electrolysis hydrogen production device under a renewable energy volatile power supply condition to realize water-electrolysis hydrogen production with high efficiency, low curtailment and high power supply variation adaptability.

Preferably, a storage and transport unit includes a transport boat commuting between the island and the land, and a green fuel receiving station, a detachable, assembleable and transportable floating high-pressure and low-temperature storage tank, an efficient and active heat-insulation system under an all-weather complex sea condition, and a safe and efficient green fuel transfer system of an unstable platform, which are arranged on land;
the transport boat is configured for transporting the green fuels generated by the green fuel synthesis unit to the green fuel receiving station and transporting the carbon dioxide captured by the carbon capture device to the green fuel synthesis unit;
the green fuel receiving station is configured for receiving the green fuels from the transport boat;
the floating high-pressure and low-temperature storage tank is configured for storing the green fuels received by the green fuel receiving station;
the green fuel transfer system is configured for realizing offshore unattended docking transfer/filling of the green fuels to transport the green fuels to the green fuel comprehensive utilization unit or to the outside;
the heat-insulation system is configured for heat insulation of the transport boat, the green fuel receiving station, the floating high-pressure and low-temperature storage tank and the transfer system.

Preferably, the green fuel receiving station includes an ammonia storage device, a methane storage device and a methanol storage device.

Preferably, the energy storage device includes one or a combination of electrochemical energy storage and thermal energy storage; and the green fuel comprehensive utilization unit includes a coal-fired power generation device, a natural gas methane power generation device, a methanol power generation device, an ammonia released energy power generation device, the natural gas methane power generation device is connected to the methane storage device of the green fuel receiving station, the methanol power generation device is connected to the methanol storage device of the green fuel receiving station, and the ammonia released energy power generation device is connected to the ammonia storage device of the green fuel receiving station.

The power generation principle of the coal-fired power generation device is as follows: coal is combusted in a boiler furnace to release heat energy to generate high-temperature smoke containing carbon dioxide; when the high-temperature smoke flows through a heated surface of a boiler, a working medium (water) is heated into high-temperature and high-pressure steam by heat exchange; the steam is transported to a steam turbine to drive multi-stage turbine blades to rotate; and a turbine rotor is mechanically connected to a generator using a coupling to convert mechanical energy into electric energy, and the electric energy is output.

The power generation principle of the natural gas methane power generation device is as follows: an air compressor pressurizes air and delivers the pressurized air into a combustion chamber, and the pressurized air and methane are mixed and ignited to generate high-temperature gas to drive a gas turbine to generate power; and then, a waste heat boiler recovers waste gas at a temperature over 500° C. discharged by the gas turbine to produce steam to drive a steam turbine to generate power again.

The power generation principle of the methanol power generation device is as follows: a methanol gas mixture is ignited by an ignition plug to push a piston to move to convert chemical energy into mechanical energy; a crankshaft synchronously drives a generator rotor to rotate using a coupling to cut a magnetic induction line to produce three-phase alternating current; and a control system controls an air-fuel ratio, an advance angle of ignition and a power generation load in real time.

The power generation principle of the ammonia released energy power generation device is as follows: ammonia and air are mixed and then combusted in a combustion chamber to release a large quantity of heat energy; and the heat energy is transferred to a working medium (such as water) using a heat exchanger to convert the working medium into steam, and the steam drives a steam turbine to generate power.

Preferably, the carbon capture device captures carbon dioxide in smoke generated by the green fuel comprehensive utilization unit using an absorption solution through a chemical absorption method.

The disclosure also relates to a sea-land resource collaborative and optimal scheduling method based on the sea-land collaboration-based multi-energy coupling low-carbon new energy system, including the following steps:
Step 1, acquiring natural resource parameters of wind energy and light energy at multiple time scales, and calculating a power output of the low-carbon power generation unit according to the natural resource parameters and a configurated capacity of the low-carbon power generation unit;
Step 2, inputting a local power load, and calculating a power surplus based on the power output of the low-carbon power generation unit and the power load; if the power surplus is greater than 0, producing green fuels using the green fuel synthesis unit to consume surplus power and/or storing surplus power using the energy storage device, and updating an energy storage capacity of the energy storage device; or, if the power surplus is less than or equal to 0, compensating for a power shortage using the energy storage device and generating green fuels using the green fuel synthesis unit, and updating the energy storage capacity of the energy storage device, wherein the produced green fuels include ammonia, methane and methanol, an operation strategy of the green fuel synthesis unit (i.e., output proportions of ammonia, methane and methanol) is set, and total carbon consumption is calculated;
Step 3, redetermining whether the power shortage still exits; if the power shortage still exits, making an operation strategy of the green fuel comprehensive utilization unit, wherein the operation strategy of the green fuel comprehensive utilization unit is set for maximizing a power output of the green fuels and minimizing a power output of carbon fuels to make up for the power shortage;
Step 4, calculating a carbon output of the green fuel comprehensive utilization unit based on the operation strategy made in Step 3, and comparing the carbon output of the green fuel comprehensive utilization unit with the total carbon consumption calculated in Step 2; if the carbon output is less than or equal to the total carbon consumption, returning to Step 3, adjusting the operation strategy of the green fuel comprehensive utilization unit, increasing the power output of the carbon fuels, and decreasing the power output of the green fuels; or, if the carbon output is greater than the total carbon consumption, calculating total carbon emission; if the total carbon emission does not satisfy emission and design requirements, returning to Step 2, adjusting the operation strategy of the green fuel synthesis unit, increasing the output proportions of methane and methanol, and deceasing the output proportion of ammonia; when the carbon output is less than or equal to the total carbon consumption and the total carbon emission satisfies the emission and design requirements, taking one of the adjusted operation strategy of the green fuel synthesis unit and the adjusted operation strategy of the green fuel comprehensive utilization unit as an operation scheduling scheme of the sea-land collaboration-based multi-energy coupling low-carbon new energy system; and
Step 5, repeating Steps 1-4 to form multiple operation scheduling schemes of the sea-land collaboration-based multi-energy coupling low-carbon new energy system, screening out an operation scheduling scheme that satisfies objective functions and constraints, obtaining a single-objective optimal operation scheduling scheme or a multi-objective optimal Pareto frontier using an optimization algorithm, and operating the sea-land collaboration-based multi-energy coupling low-carbon new energy system based on the single-objective optimal operation scheduling scheme or the multi-objective optimal Pareto frontier.

Preferably, the operation scheduling schemes formed in Step 4 satisfy the following balances:
island hydrogen satisfies the following balance:

$${n}_{{H}_{2} , prod}^{island} ( t ) - {n}_{{H}_{2} , {NH}_{3}}^{island} ( t ) - {n}_{{H}_{2} , {CH}_{4}}^{island} ( t ) - {n}_{{H}_{2} , {CH}_{3} OH}^{island} ( t ) - {n}_{{H}_{2} , charge}^{island} ( t ) + {n}_{{H}_{2} , discharge}^{island} ( t ) = 0$$

where, n_{H}_{₂}_{,prod}^{island}(t) is a production output of hydrogen, n_{H}_{₂}_{,NH}_{₃}^{island}(t) is a quantity of hydrogen consumed for ammonia production, n_{H}_{₂}_{,CH}_{₄}^{island}(t) is a quantity of hydrogen consumed for methane production, n_{H}_{₂}_{,CH}_{₃}_{OH}^{island}(t) is a quantity of hydrogen consumed for methanol production, n_{H}_{₂}_{,charge}^{island}(t) is a quantity of hydrogen stored in an island hydrogen storage device, and n_{H}_{₂}_{,discharge}^{island}(t) is a quantity of hydrogen discharged from the island hydrogen storage device;
ammonia satisfies the following balance:

$${n}_{N {H}_{3} , prod}^{island} ( t ) - {n}_{N {H}_{3} , charge}^{b o a t} ( t ) {-}_{N {H}_{3} , cb}^{island} ( t ) - {n}_{N {H}_{3} , charge}^{island} ( t ) + {n}_{N {H}_{3} , discharge}^{island} ( t ) = 0 ,$$

where, n_{NH}_{₃}_{,prod}^{island}(t) is a production output of ammonia, n_{NH}_{₃}_{,charge}^{boat}(t) is a quantity of ammonia loaded onto a boat, n_{NH}_{₃}_{,cb}^{island}(t) is a quantity of ammonia combusted on an island, n_{NH}_{₃}_{,charge}^{island}(t) is a quantity of ammonia stored in an island ammonia storage device, and n_{NH}_{₃}_{,discharge}^{island}(t) is a quantity of ammonia discharged from the island ammonia storage device;
methane satisfies the following balance:

$${n}_{C {H}_{4} , prod}^{island} ( t ) - {n}_{C {H}_{4} , charge}^{b o a t} ( t ) - {n}_{C {H}_{4} , cb}^{island} ( t ) - {n}_{C {H}_{4} , charge}^{island} ( t ) + {n}_{C {H}_{4} , discharge}^{island} ( t ) = 0 ,$$

where, n_{CH}_{₄}_{,prod}^{island}(t) is a production output of methane, n_{CH}_{₄}_{,charge}^{boat}(t) is a quantity of methane loaded onto the boat, n_{CH}_{₄}_{,cb}^{island}(t) is a quantity of methane combusted on the island, n_{CH}_{₄}_{,charge}^{island}(t) is a quantity of methane stored in an island methane storage device, and n_{CH}_{₄}_{,discharge}^{island}(t) is a quantity of methane discharged from the island methane storage device;
methanol satisfies the following balance:

$${n}_{C {H}_{3} OH , prod}^{island} ( t ) - {n}_{C {H}_{3} OH , charge}^{b o a t} ( t ) - {n}_{C {H}_{3} OH , cb}^{island} ( t ) - {n}_{C {H}_{3} OH , charge}^{island} ( t ) + {n}_{C {H}_{3} OH , discharge}^{island} ( t ) = 0 ,$$

where, n_{CH}_{₃}_{OH,prod}^{island}(t) is a production output of methanol, n_{CH}_{₃}_{OH,charge}^{boat}(t) is a quantity of methanol loaded onto the boat, n_{CH}_{₃}_{OH,cb}^{island}(t) is a quantity of methanol combusted on the island, n_{CH}_{₃}_{OH,charge}^{island}(t) is a quantity of methanol stored in an island methanol storage device, and n_{CH}_{₃}_{OH,discharge}^{island}(t) is a quantity of methanol discharged from the island methanol storage device;
carbon dioxide satisfies the following balance:

$${n}_{C {O}_{2} , {CH}_{4}}^{island} ( t ) + {n}_{C {O}_{2} , {CH}_{3} O H}^{island} ( t ) + {n}_{C {O}_{2} , coal}^{island} ( t ) - {n}_{C {O}_{2} , capture}^{island} ( t ) + {n}_{{CO}_{2} , discharge}^{b o a t} - {n}_{C {O}_{2} , emission}^{island} ( t ) = 0 ,$$

where, n_{CO}_{₂}_{,CH}_{₄}^{island}(t) is a quantity of carbon dioxide produced by methane combustion, n_{CO}_{₂}_{,CH}_{₃}_{OH}^{island}(t) is a quantity of carbon dioxide produced by methanol combustion, island n_{CO}_{₂}_{,coal}^{island}(t) is a quantity of carbon dioxide produced by coal combustion, n_{CO}_{₂}_{,capture}^{island}(t) is a quantity of carbon dioxide captured by an island carbon capture facility, n_{CO}_{₂}_{,discharge}^{boat}(t) is a quantity of carbon dioxide discharged by the boat, and n_{CO}_{₂}_{,emission}^{island}(t) is a quantity of carbon dioxide directly discharged by the island;
power satisfies the following balance:

$${P}_{w i n d}^{island} ( t ) + {P}_{solar}^{island} ( t ) + {P}_{N {H}_{3} , cb}^{island} ( t ) + {P}_{C {H}_{4} , cb}^{island} ( t ) + {P}_{C {H}_{3} OH , cb}^{island} ( t ) - {P}_{{H}_{2} , prod}^{island} ( t ) - {P}_{N {H}_{3} , prod}^{island} ( t ) - {P}_{C {H}_{4} , prod}^{island} ( t ) - {P}_{C {H}_{3} OH , prod}^{island} ( t ) = {P}_{load}^{island} ( t ) ,$$

where, P_{wind}^{island}(t), Pₛₒₗₐᵣ^{island}(t), P_{NH}_{₃}_{,cb}^{island}(t), P_{CH}_{₄}_{,cb}^{island}(t) and P_{CH}_{₃}_{OH,cb}^{island}(t) are respectively power of a wind power generator, power of a solar power generator, power of an ammonia power generator, power of a methane power generator and power of a methanol power generator; P_{H}_{₂}_{,prod}^{island}(t), P_{NH}_{₃}_{,prod}^{island}(t), P_{CH}_{₄}_{,prod}^{island}(t) and P_{CH}_{₃}_{OH,prod}^{island}(t) are respectively power consumption of hydrogen production, power consumption of ammonia production, power consumption of methane production and power consumption of methanol production; P_{load}^{island}(t) is a real-time power load of the island.

Preferably, the constraints in Step 5 include:
a constraint of an ammonia synthesis process of an electro-catalytic ammonia production device, a formula of which is:

$$3 {n}_{{H}_{2} , {NH}_{3}}^{island} ( t ) + {n}_{{N}_{2}}^{island} ( t ) \to 2 {n}_{N {H}_{3} , prod}^{island} ( t ) ,$$

where, n_{H}_{₂}_{,NH}_{₃}^{island}(t) is a quantity of hydrogen consumed for ammonia production, n_{N}_{₂}^{island}(t) is a quantity of nitrogen consumed for ammonia production, and n_{NH}_{₃}_{,prod}^{island}(t) is a production output of ammonia;
a constraint of a methane synthesis process of a methane synthesis device, a formula of which is:

$$4 {n}_{{H}_{2} , {CH}_{4}}^{island} ( t ) + {n}_{C {O}_{2} , {CH}_{4}}^{island} ( t ) \to {n}_{C {H}_{4} , prod}^{island} ( t ) + 2 {H}_{2} O ,$$

where, n_{H}_{₂}_{,CH}_{₄}^{island}(t) is a quantity of hydrogen consumed for methane production, n_{CO}_{₂}_{,CH}_{₄}^{island}(t) is a quantity of carbon dioxide generated by methane combustion, and n_{CH}_{₄}_{,prod}^{island}(t) is a production output of methane;
a constraint of a methanol synthesis process of a methanol synthesis device, a formula of which is:

$$2 {n}_{{H}_{2} , {CH}_{3} OH}^{island} ( t ) + {n}_{C {O}_{2} , {CH}_{3} O H}^{island} ( t ) \to {n}_{C {H}_{3} OH , prod}^{island} ( t ) + {H}_{2} O ,$$

where, n_{H}_{₂}_{,CH}_{₃}_{OH}^{island}(t) is a quantity of hydrogen consumed for methanol production, island n_{CO}_{₂}_{,CH}_{₃}_{OH}^{island}(t) is a quantity of carbon dioxide consumed for methanol production, and n_{CH}_{₃}_{OH,prod}^{island}(t) is a production output of methanol;
energy consumption constraints for green fuel production, formulas of which are:

$$\begin{matrix}{P}_{{H}_{2} , prod}^{island} ( t ) = {f}_{{H}_{2} , prod}^{island} ( {n}_{{H}_{2} , prod}^{island} ( t ) ) , \\ {P}_{{H}_{3} , prod}^{island} ( t ) = {f}_{{H}_{3} , prod}^{island} ( {n}_{{H}_{3} , prod}^{island} ( t ) ) , \\ {P}_{{CH}_{4} , prod}^{island} ( t ) = {f}_{{CH}_{4} , prod}^{island} ( {n}_{{CH}_{4} , prod}^{island} ( t ) ) , \\ {P}_{{CH}_{3} OH , prod}^{island} ( t ) = {f}_{{CH}_{3} OH , prod}^{island} ( {n}_{{CH}_{3} OH , prod}^{island} ( t ) ) ,\end{matrix}$$

where, f_{H}_{₂}_{,prod}^{island}, f_{NH}_{₃}_{,prod}^{island}, f_{CH}_{₄}_{,prod}^{island} and f_{CH}_{₃}_{OH,prod}^{island} are respectively energy consumption conversion relations in a green fuel production process; P_{H}_{₂}_{,prod}^{island}(t), P_{NH}_{₃}_{,prod}^{island}(t), P_{CH}_{₄}_{,prod}^{island}(t) and P_{CH}_{₃}_{OH,prod}^{island}(t) are respectively power consumption of hydrogen production, power consumption of ammonia production, power consumption of methane production and power consumption of methanol production at a time t; n_{H}_{₂}_{,prod}^{island}(t), n_{NH}_{₃}_{,prod}^{island}(t), n_{CH}_{₄}_{,prod}^{island}(t) and island n_{CH}_{₃}_{OH,prod}^{island}(t) are respectively production outputs of hydrogen, ammonia, methane and methanol at the time t;
energy consumption constraints of green fuel combustion, formulas of which are:

$$\begin{matrix}{P}_{{NH}_{3} , cb}^{island} ( t ) = {f}_{{NH}_{3} , cb}^{island} ( {n}_{{NH}_{3} , cb}^{island} ( t ) ) , \\ {P}_{{CH}_{4} , cb}^{island} ( t ) = {f}_{{CH}_{4} , cb}^{island} ( {n}_{{CH}_{4} , cb}^{island} ( t ) ) , \\ {P}_{{CH}_{3} OH , cb}^{island} ( t ) = {f}_{{CH}_{3} OH , cb}^{island} ( {n}_{{CH}_{3} OH , cb}^{island} ( t ) ) ,\end{matrix}$$

where, f_{NH}_{₃}_{,cb}^{island}, f_{CH}_{_{4,cb}}^{island} and f_{CH}_{₃}_{OH,cb}^{island} are respectively energy consumption conversion relations in a utilization process of island green fuels; P_{NH}_{₃}_{,cb}^{island}(t), P_{CH}_{₄}_{,cb}^{island}(t) and P_{CH}_{₃}_{OH,cb}^{island}(t) are respectively power of an ammonia power generator, power of a methane power generator and power of a methanol power generator at the time t; n_{NH}_{₃}_{,cb}^{island}(t), n_{CH}_{₄}_{,cb}^{island}(t) and n_{CH}_{₃}_{OH,cb}^{island}(t) are respectively a quantity of ammonia combusted on an island, a quantity of methane combusted on the island and a quantity of methanol combusted on the island;
a capacity constraint of an island hydrogen storage tank, a formula of which is:

$$\frac{( {n}_{{H}_{2} , charge}^{island} ( t ) - {n}_{{H}_{2} , discharge}^{island} ( t ) ) \times Δ t}{{Capacity}_{{H}_{2}}^{island}} = {SOC}_{{H}_{2}}^{island} ( t ) - S O {C}_{{H}_{2}}^{island} ( t - 1 ) ,$$

where, n_{H}_{₂}_{,charge}^{island}(t) and n_{H}_{₂}_{,discharge}^{island}(t) are respectively a quantity of hydrogen stored in the island hydrogen storage tank and a quantity of hydrogen discharged from the island hydrogen storage tank at the time t, Capacity_{H}_{₂}^{island} is a total capacity of the island hydrogen storage tank, and SOC_{H}_{₂}^{island}(t) and SOC_{H}_{₂}^{island}(t−1) are respectively a hydrogen storage capacity of the island hydrogen storage tank at the time t and a hydrogen storage capacity of the island hydrogen storage tank at a time (t−1);
a capacity constraint of a methane storage tank of a transport boat, a formula of which is:

$$\frac{( {n}_{{CH}_{4} , charge}^{boat} ( t ) - {n}_{{CH}_{4} , discharge}^{boat} ( t ) ) \times Δ t}{{Capacity}_{{CH}_{4}}^{boat}} = {SOC}_{{CH}_{4}}^{boat} ( t ) - S O {C}_{{CH}_{4}}^{boat} ( t - 1 ) ,$$

where, n_{CH}_{₄}_{,charge}^{boat}(t) and n_{CH}_{₄}_{,discharge}^{boat}(t) are respectively a quantity of methane stored in the methane storage tank of the transport boat and a quantity of methane discharged from the methane storage tank of the transport boat at the time t, Capacity_{CH}_{₄}^{boat} is a total capacity of the methane storage tank, and SOC_{CH}_{₄}^{boat}(t) and SOC_{CH}_{₄}^{boat}(t−1) are respectively a capacity of the methane storage tank of the transport boat at the time t and a capacity of the methane storage tank of the transport boat at the time (t−1);
a capacity constraint of a carbon dioxide storage tank of the transport boat, a formula of which is:

$$\frac{( {n}_{{CO}_{2} , charge}^{boat} ( t ) - {n}_{{CO}_{2} , discharge}^{boat} ( t ) ) \times Δ t}{{Capacity}_{{CO}_{2}}^{boat}} = {SOC}_{{CO}_{2}}^{boat} ( t ) - S O {C}_{{CO}_{2}}^{boat} ( t - 1 ) ,$$

where, n_{CO}_{₂}_{,charge}^{boat}(t) and n_{CO}_{₂}_{,discharge}^{boat}(t) are respectively a quantity of carbon dioxide stored in the carbon dioxide storage tank of the transport boat and a quantity of carbon dioxide discharged from the carbon dioxide storage tank of the transport boat at the time t, Capacity_{CO}_{₂}^{boat} is a total capacity of the carbon dioxide storage tank, and SOC_{CO}_{₂}^{boat}(t) and SOC_{CO}_{₂}^{boat}(t−1) are respectively a capacity of the carbon dioxide storage tank of the transport boat at the time t and a capacity of the carbon dioxide storage tank of the transport boat at the time (t−1);
an operating state constraint of the transport boat, a formula of which is:

$${I}_{c h a r g e} ( t ) + {I}_{d i s c h a r g e} ( t ) + {I}_{sailing} ( t ) + {I}_{m o o r i n g} ( t ) = 1 ,$$

where, I_{charge}(t) indicates that the boat is in a working medium charging state at the time t, I_{discharge}(t) indicates that the boat is in a working medium discharging state, I_{sailing}(t) indicates that the boat is in a sailing state, and I_{mooring}(t) indicates that the boat is in a mooring state.

Preferably, the objective functions in Step 5 include:
an operating cost objective function, a formula of which is:

$$cost ( t ) = ( {n}_{C {O}_{2} , emission}^{island} ( t ) + {n}_{C {O}_{2} , e m i s s i o n}^{land} ( t ) ) \times {Price}_{C {O}_{2}} + {n}_{coal , cb}^{land} ( t ) \times {Price}_{coal}$$

where, Price_{CO}_{₂} is a carbon sink price, Price_{coal} is a coal price, n_{CO}_{₂}_{,emission}^{island} is a quantity of carbon dioxide directly discharged by an island, n_{CO}_{₂}_{,emission}^{land}(t) is carbon dioxide emission of land, and n_{coal,cb}^{land}(t) is coal combustion of the land;
an operating revenue objective function, a formula of which is:

$$revenue ( t ) = ( {P}_{load}^{island} ( t ) + {P}_{d e m a n d}^{land} ( t ) ) \times {Price}_{electricity} ,$$

where, revenue(t) is the operating revenue objective function, and P_{load}^{island}(t) and P_{demand}^{land}(t) are respectively a power load of the island at a time t and a power demand of the island at the time t; Price_{electricity} is an electricity price.

Preferably, in Step 4, a surplus of the green fuels is calculated according to the operation strategy of the green fuel synthesis unit and the operation strategy of the green fuel comprehensive utilization unit, and the calculated surplus is taken as a quantity of green fuels transported to the outside.

The objective functions or constraints in Step 5 include a levelized cost of energy for evaluating economy, carbon emission per kilowatt-hour for evaluating environmental influences, and a load satisfaction rate for evaluating power supply reliability; and the optimization algorithm is a genetic algorithm or a particle swarm algorithm, or is solved using a linear programming solver.

Compared with the prior art, the technical solution of the disclosure has the following beneficial effects:

According to the sea-land collaboration-based multi-energy coupling low-carbon new energy system provided by the disclosure, the low-carbon power generation unit is constructed on a sea and/or an island, power is generated using abundant and stable solar energy and wind energy on the sea and the island, hydrogen and ammonia are prepared using seawater, and the green fuel synthesis unit prepares green fuels using the prepared hydrogen and carbon dioxide produced by combustion of coal and natural gas in the green fuel comprehensive utilization unit. The system makes full use of abundant and stable solar energy, wind energy and seawater resources on the sea and the island to produce green fuels to be used by the green fuel comprehensive utilization unit, such that the use of coal and natural gas in the green fuel comprehensive utilization unit is reduced; meanwhile, produced carbon dioxide is used as raw materials to prepare green fuels again, such that the emission of harmful gas and carbon dioxide is reduced.

According to the sea-land collaborative low-carbon intelligent control method provided by the disclosure, an operation strategy model of the green fuel synthesis unit and an operation strategy model of the green fuel comprehensive utilization unit are constructed, the operation strategy of the green fuel comprehensive utilization unit is made based on a power output of the low-carbon power generation unit and a power load, the carbon emission is calculated based on the operation strategy of the green fuel comprehensive utilization unit, the operation strategy of the green fuel synthesis unit is adjusted based on the carbon emission, and operation scheduling schemes satisfying the carbon emission standard and using a small quantity of coal are obtained; an operation scheduling scheme that satisfies constraints is screened out based on objective functions or constraints of multiple operation scheduling schemes, a single-objective optimal operation scheduling scheme or a multi-objective optimal Pareto frontier is obtained using an optimization algorithm, and then a safe and reliable operating mode for multi-form and flexible resources of an island-type multi-energy flow coupling system (sending-end offshore island/isolated distant island) is provided, the sea-land collaborative scheduling capacity of the multi-energy flow coupling system may be studied and determined according to the variation trend of supply and demand, collaborative scheduling at multiple time scales such as day, hour and minute is realized, and the comprehensive energy utilization rate of the system is increased by over 30%.

### BRIEF DESCRIPTION OF THE DRAWINGS

FIG. 1 is a schematic structural diagram of a sea-land collaboration-based multi-energy coupling low-carbon new energy system according to the disclosure; and

FIG. 2 is a schematic operation flow diagram of a sea-land collaboration-based multi-energy coupling low-carbon new energy system.

### DETAILED DESCRIPTION OF PREFERRED EMBODIMENTS

The disclosure is described in detail below, and the technical solutions in the embodiments of the disclosure are clearly and completely described. Obviously, the embodiments described below are merely illustrative ones, and are not all possible ones of the disclosure. All other embodiments obtained by those ordinarily skilled in the art based on the following ones without creative labor should also fall within the protection scope of the disclosure.

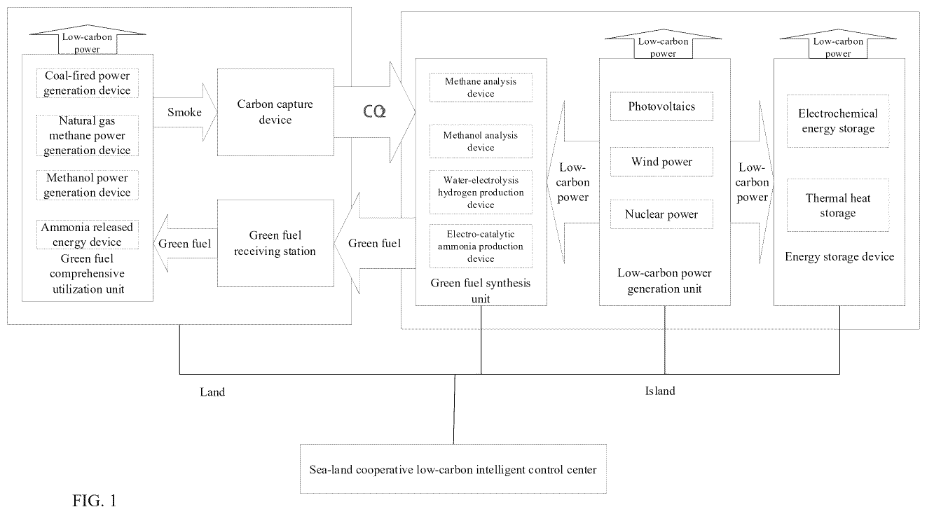

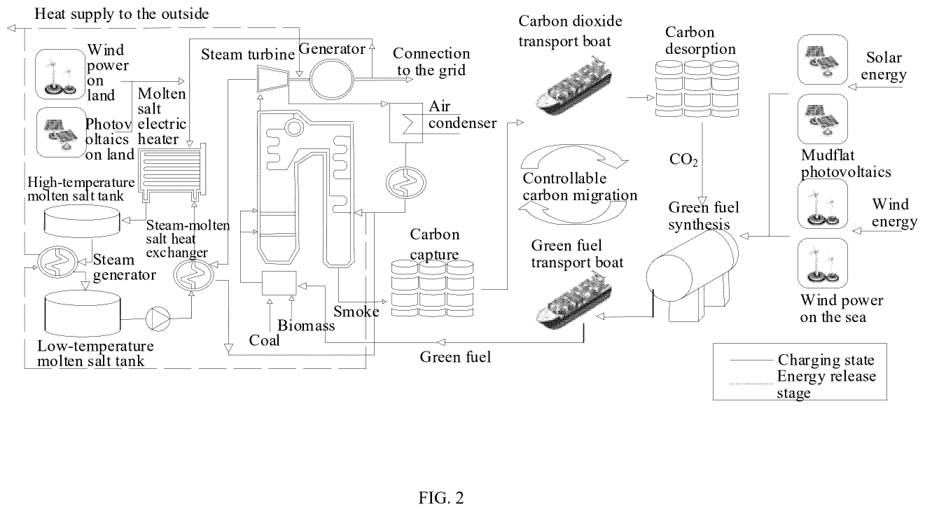

Referring to FIGS. 1 and 2, a sea-land collaboration-based multi-energy coupling low-carbon new energy system according to the disclosure includes a low-carbon power generation unit, a green fuel synthesis unit and an energy storage device which are arranged on a sea and an island, a green fuel comprehensive utilization unit and a carbon capture device which are arranged on the island and/or on land, and a multi-energy flow coupling-based sea-land collaborative low-carbon intelligent control center arranged on the island or on land.

The low-carbon power generation unit includes one or a combination of more than one of photovoltaic power generation, wind power generation and nuclear power generation and generates zero-carbon/low-carbon power based on wind, light and nuclear resources.

The green fuel synthesis unit produces hydrogen and ammonia using the zero-carbon/low-carbon power generated by the low-carbon power generation unit and produces methane and methanol using the hydrogen and carbon dioxide from the carbon capture device, and the ammonia, the methane and the methanol are used by the green fuel comprehensive utilization unit as green fuels. The green fuel synthesis unit includes: a seawater-electrolysis hydrogen production device driven by a wind-light volatile power supply, an electro-catalytic ammonia production device, a methane synthesis device and a methanol synthesis device.

The seawater-electrolysis hydrogen production device driven by the wind-light volatile power supply is configured for producing hydrogen by seawater electrolysis, and a seawater-electrolysis hydrogen production catalyst of a multi-stage array structure and a photovoltaic/wind power volatile renewable energy-based hydrogen production control system are arranged in the seawater-electrolysis hydrogen production device. The photovoltaic/wind power volatile renewable energy-based hydrogen production control system analyzes and optimizes reaction performance of the seawater-electrolysis hydrogen production device using an electrochemical-heat-fluid coupling numerical model of an electrolytic bath and adjusts in real time operation control of the seawater-electrolysis hydrogen production device under a renewable energy volatile power supply condition. The surface of an electrode used by the seawater-electrolysis hydrogen production device is covered with an alloy catalyst modified by mixing multiple elements, doped precious metal elements include one or more of nickel, ruthenium, cadmium, molybdenum and platinum, a doped metal loading capacity of an electrode catalyst is less than 0.5 mg/cm², an cathodic overpotential is less than or equal to 300 mV@1000 mA/cm², and an anodic overpotential is less than or equal to 600 mV@1000 mA/cm².

The electro-catalytic ammonia production device is configured for synthesizing ammonia by hydrogen and nitrogen. An electro-catalytic material in the seawater-electrolysis hydrogen production device is a metal organic complex, a cathodic material is an Al—N₂ or Zr—N₂ cathodic material, and an electrolyte solution is one or more of ionic liquid, ionic liquid/organic solvent, and ionic liquid/organic solvent/water. The electro-catalytic ammonia production device realizes mass production of green ammonia using a multi-stage serial mode of the electrolytic bath, a partial current density of electro-catalysis ammonia N₂ production system is over 300 mA cm⁻², the ammonia yield reaches 5×10⁻⁵ mol·s⁻¹·cm⁻², and the catalytic efficiency after 500 h of electro-catalysis is maintained over 90%.

The methanol synthesis device prepares a methanol catalyst by selective CO₂ hydrogenation using high-activity and high-yield substances under a low-temperature and low-pressure condition, and using a novel reaction-separation coupled CO₂ hydrogenation reactor, the conversion rate per pass of carbon dioxide is greater than or equal to 25%, the total conversion rate of hydrogen is greater than or equal to 92%, the total selectivity of methanol is greater than or equal to 96%, and the content of methanol in an organic phase is greater than or equal to 99%.

The energy storage device performs charging using surplus power in a case where power is still surplus after the low-carbon power generation unit satisfies a power load, and compensates for a power shortage in a case where the low-carbon power generation fails to satisfy the power load. The energy storage device includes one or a combination of electrochemical energy storage and thermal energy storage.

The green fuel comprehensive utilization unit includes one or a combination of a coal-fired power generation device, a natural gas methane power generation device, a methanol power generation device, an ammonia released energy power generation device, the natural gas methane power generation device is connected to a methane storage device of a green fuel receiving station, the methanol power generation device is connected to a methanol storage device of the green fuel receiving station, and the ammonia released energy power generation device is connected to an ammonia storage device of the green fuel receiving station.

The green fuel comprehensive utilization unit generates power preferentially using the green fuels in a case where the power load is still not satisfied after the energy storage device compensates for the power shortage, and generates power by combusting natural gas and/or coal in a case where the power load is still not satisfied after the green fuel comprehensive utilization unit generates power using the green fuels. The green fuel comprehensive utilization unit generates steam by combusting the green fuels or carbon fuels and drives a steam turbine to operate, the steam turbine drives a generator to generate power, heat is supplied to the outside using the generated steam; if heat is still surplus after being supplied to the outside, surplus heat is stored in a molten salt tank; when heat supplied to the outside is insufficient, heat stored in the molten salt tank acts on the steam generator to generate steam to make up for a heat deficiency, at this moment, steam generated by the green fuel comprehensive utilization unit will not heat molten salt in the molten salt tank, and the temperature in the molten salt tank deceases; and when the temperature reaches a set lower limit, electric energy generated by the green fuel comprehensive utilization unit may be used to heat the molten salt in the molten salt tank by electric heating.

The carbon capture device is configured for capturing carbon dioxide produced by the green fuel comprehensive utilization unit and transporting the carbon dioxide into the green fuel synthesis unit. Specifically, the carbon capture device captures carbon dioxide in smoke produced by the green fuel comprehensive utilization unit using absorption liquid through a chemical absorption method, separates the carbon dioxide from the absorption liquid using a carbon desorption method and supplies the carbon dioxide to the green fuel compressive utilization unit.

The sea-land collaborative low-carbon intelligent control center calculates a zero-carbon and/or low-carbon power generation capacity according to acquired parameters of the wind energy, light energy and nuclear energy on the sea and/or the island, and controls operation strategies of the green fuel synthesis unit and the green fuel comprehensive utilization unit according to a power output of the energy storage device and the power load.

The sea-land collaboration-based multi-energy coupling low-carbon new energy system further includes a storage and transport unit suitable for a complex sea condition. The storage and transport unit includes a transport boat commuting between the island and the land, and a green fuel receiving station, a detachable, assembleable and transportable floating high-pressure and low-temperature storage tank, an efficient and active heat-insulation system under an all-weather complex sea condition, and a safe and efficient green fuel transfer system of an unstable platform, which are arranged on land. The transport boat is configured for transporting the green fuels generated by the green fuel synthesis unit to the green fuel receiving station and transporting the carbon dioxide captured by the carbon capture device to the green fuel synthesis unit to realize controllable carbon migration. The green fuel receiving station is configured for receiving the green fuels from the transport boat. The floating high-pressure and low-temperature storage tank is configured for storing the green fuels received by the green fuel receiving station. The green fuel transfer system is configured for realizing offshore unattended docking transfer/filling of the green fuels to transport the green fuels to the green fuel comprehensive utilization unit or to the outside. The heat-insulation system is configured for heat insulation of the transport boat, the green fuel receiving station, the floating high-pressure and low-temperature storage tank and the transfer system.

The disclosure further relates to a sea-land resource collaborative and optimal scheduling method, including the following steps:

Step 1, natural resource parameters of wind energy and light energy at multiple time scales are acquired, and a power output of a low-carbon power generation unit is calculated according to the natural resource parameters and a configurated capacity of the low-carbon power generation unit.

Step 2, a local power load is input, and a power surplus P₁ₛᵤᵣₚₗᵤₛ is calculated based on the power output of the low-carbon power generation unit and the power load, wherein a calculation formula is:

$${P}_{1 surplus} = P ( t ) - {P}_{d e m a n d} ( t ) \qquad ( 1 )$$

where, P(t) is a power output of the low-carbon power generation unit at a time t, and P_{demand}(t) is a power load at the time t;
if the power surplus is greater than 0, green fuels are produced using a green fuel synthesis unit to consume surplus power and/or surplus power is stored using an energy storage device, and an energy storage capacity of the energy storage device is updated; or, if the power surplus is less than or equal to 0, a power shortage is compensated using the energy storage device, green fuels are generated using the green fuel synthesis unit, and the energy storage capacity of the energy storage device is updated, wherein the produced green fuels include ammonia, methane and methanol, an operation strategy of the green fuel synthesis unit (i.e., output proportions of ammonia, methane and methanol) is set, and total carbon consumption is calculated.

Step 3, the power surplus P₁ₛᵤᵣₚₗᵤₛ is recalculated to determine whether the power shortage still exits, wherein a calculation formula is:

$${P}_{2 surplus} = P ( t ) + {P}_{storage} ( t ) - {P}_{synthesis} ( t ) - {P}_{demand} ( t ) , \qquad ( 2 )$$

where, P_{storage}(t) is a power output of the energy storage device in Step 2; when the energy storage device does not need to make up for the power shortage, P_{storage}(t)=0; P_{synthesis}(t) is power consumed for green fuel combustion;
if the power shortage still exits, an operation strategy of the green fuel comprehensive utilization unit is made, wherein the operation strategy of the green fuel comprehensive utilization unit is set for maximizing a power output of the green fuels and minimizing a power output of carbon fuels to make up for the power shortage, and the carbon fuels refer to coal and/or natural gas.

Step 4, a carbon output of the green fuel comprehensive utilization unit is calculated based on the operation strategy made in Step 3, and the carbon output of the green fuel comprehensive utilization unit is compared with the total carbon consumption calculated in Step 2; if the carbon output is less than or equal to the total carbon consumption, Step 3 is performed, the operation strategy of the green fuel comprehensive utilization unit is adjusted, the power output of the carbon fuels is increased, and the power output of the green fuels is decreased; or, if the carbon output is greater than the total carbon consumption, total carbon emission is calculated; if the total carbon emission does not satisfy emission and design requirements, Step 2 is performed, the operation strategy of the green fuel synthesis unit is adjusted, the output proportions of methane and methanol are increased, and the output proportion of ammonia is decreased; when the carbon output is less than or equal to the total carbon consumption and the total carbon emission satisfies the emission and design requirements, one of the adjusted operation strategy of the green fuel synthesis unit and the adjusted operation strategy of the green fuel comprehensive utilization unit is taken as an operation scheduling scheme of the multi-energy coupling low-carbon new energy system; wherein, in this step, a surplus of the green fuels is calculated according to the operation strategy of the green fuel synthesis unit and the operation strategy of the green fuel comprehensive utilization unit, and the calculated surplus is taken as a quantity of green fuels transported to the outside.

Based on the above steps, multiple operation scheduling schemes of the sea-land collaboration-based multi-energy coupling low-carbon new energy system are formed, and the operation scheduling schemes satisfy the following balances:
island hydrogen satisfies the following balance:

$${n}_{{H}_{2} , prod}^{island} ( t ) - {n}_{{H}_{2} , {NH}_{3}}^{island} ( t ) - {n}_{{H}_{2} , {CH}_{4}}^{island} ( t ) - {n}_{{H}_{2} , {CH}_{3} OH}^{island} ( t ) - {n}_{{H}_{2} , charge}^{island} ( t ) + {n}_{{H}_{2} , discharge}^{island} ( t ) = 0 ,$$

where, n_{H}_{₂}_{,prod}^{island}(t) is a production output of hydrogen, n_{H}_{₂}_{,NH}_{₃}^{island}(t) is a quantity of hydrogen consumed for ammonia production, n_{H}_{₂}_{,CH}_{₄}^{island}(t) is a quantity of hydrogen consumed for methane production, n_{H}_{₂}_{,CH}_{₃}_{OH}^{island}(t) is a quantity of hydrogen consumed for methanol production, n_{H}_{₂}_{,charge}^{island}(t) is a quantity of hydrogen stored in an island hydrogen storage device, and n_{H}_{₂}_{,discharge}^{island}(t) is a quantity of hydrogen discharged from the island hydrogen storage device;
ammonia satisfies the following balance:

$${n}_{N {H}_{3} , prod}^{island} ( t ) - {n}_{N {H}_{3} , charge}^{b o a t} ( t ) - {n}_{N {H}_{3} , cb}^{island} ( t ) - {n}_{N {H}_{3} , charge}^{island} ( t ) + {n}_{N {H}_{3} , discharge}^{island} ( t ) = 0 ,$$

where, n_{NH}_{₃}_{,prod}^{island}(t) is a production output of ammonia, n_{NH}_{₃}_{,charge}^{boat}(t) is a quantity of ammonia loaded onto a boat, n_{NH}_{₃}_{,cb}^{island}(t) is a quantity of ammonia combusted on an island, n_{NH}_{₃}_{,charge}^{island}(t) is a quantity of ammonia stored in an island ammonia storage device, and n_{NH}_{₃}_{,discharge}^{island}(t) is a quantity of ammonia discharged from the island ammonia storage device;
methane satisfies the following balance:

$${n}_{C {H}_{4} , prod}^{island} ( t ) - {n}_{C {H}_{4} , charge}^{b o a t} ( t ) - {n}_{C {H}_{4} , cb}^{island} ( t ) - {n}_{C {H}_{4} , charge}^{island} ( t ) + {n}_{C {H}_{4} , discharge}^{island} ( t ) = 0 ,$$

where, n_{CH}_{₄}_{,prod}^{island}(t) is a production output of methane, n_{CH}_{₄}_{,charge}^{boat}(t) is a quantity of methane loaded onto the boat, n_{CH}_{₄}_{,cb}^{island}(t) is a quantity of methane combusted on the island, n_{CH}_{₄}_{,charge}^{island}(t) is a quantity of methane stored in an island methane storage device, and n_{CH}_{₄}_{,discharge}^{island}(t) is a quantity of methane discharged from the island methane storage device;
methanol satisfies the following balance:

$${n}_{C {H}_{3} OH , prod}^{island} ( t ) - {n}_{C {H}_{3} OH , charge}^{boat} ( t ) - {n}_{C {H}_{3} OH , cb}^{island} ( t ) - {n}_{C {H}_{3} OH , charge}^{island} ( t ) + {n}_{C {H}_{3} O H , d i s c h a r g e}^{island} ( t ) = 0 ,$$

where, n_{CH}_{₃}_{OH,prod}^{island}(t) is a production output of methanol, n_{CH}_{₃}_{OH,charge}^{boat}(t) is a quantity of methanol loaded onto the boat, n_{CH}_{₃}_{OH,cb}^{island}(t) is a quantity of methanol combusted on the island, n_{CH}_{₃}_{OH,charge}^{island}(t) is a quantity of methanol stored in an island methanol storage device, and n_{CH}_{₃}_{OH,discharge}^{island}(t) is a quantity of methanol discharged from the island methanol storage device;
carbon dioxide satisfies the following balance:

$${n}_{C {O}_{2} , {CH}_{4}}^{island} ( t ) + {n}_{C {O}_{2} , {CH}_{3} O H}^{island} ( t ) + {n}_{C {O}_{2} , coal}^{island} ( t ) - {n}_{C {O}_{2} , capture}^{island} ( t ) + {n}_{{CO}_{2} , discharge}^{boat} ( t ) - {n}_{{CO}_{2} , emission}^{island} ( t ) = 0 ,$$

where, n_{CO}_{₂}_{,CH}_{₄}^{island}(t) is a quantity of carbon dioxide produced by methane combustion, n_{CO}_{₂}_{,CH}_{₃}_{OH}^{island}(t) is a quantity of carbon dioxide produced by methanol combustion, island n_{CO}_{₂}_{,coal}^{island}(t) is a quantity of carbon dioxide produced by coal combustion, n_{CO}_{₂}_{,capture}^{island}(t) is a quantity of carbon dioxide captured by an island carbon capture facility, n_{CO}_{₂}_{,discharge}^{boat}(t) is a quantity of carbon dioxide discharged by the boat, and n_{CO}_{₂}_{,emission}^{island}(t) is a quantity of carbon dioxide directly discharged by the island;
power satisfies the following balance:

$${P}_{w i n d}^{island} ( t ) + {P}_{solar}^{island} ( t ) + {P}_{N {H}_{3} , cb}^{island} ( t ) + {P}_{C {H}_{4} , cb}^{island} ( t ) + {P}_{C {H}_{3} OH , cb}^{island} ( t ) - {P}_{{H}_{2} , prod}^{island} ( t ) - {P}_{N {H}_{3} , prod}^{island} ( t ) - {P}_{C {H}_{4} , prod}^{island} ( t ) - {P}_{C {H}_{3} OH , prod}^{island} ( t ) = {P}_{load}^{island} ( t ) ,$$

where, P_{wind}^{island}(t), Pₛₒₗₐᵣ^{island}(t), P_{NH}_{₃}_{,cb}^{island}(t), P_{CH}_{₄}_{,cb}^{island}(t) and P_{CH}_{₃}_{OH,cb}^{island}(t) are respectively power of a wind power generator, power of a solar power generator, power of an ammonia power generator, power of a methane power generator and power of a methanol power generator; P_{H}_{₂}_{,prod}^{island}(t), P_{NH}_{₃}_{,prod}^{island}(t), P_{CH}_{₄}_{,prod}^{island}(t) and P_{CH}_{₃}_{OH,prod}^{island}(t) are respectively power consumption of hydrogen production, power consumption of ammonia production, power consumption of methane production and power consumption of methanol production; P_{load}^{island}(t) is a real-time power load of the island.

Step 5: based on the multiple operation scheduling schemes of the sea-land collaboration-based multi-energy coupling low-carbon new energy system formed in Step 4, objective functions or constrains of the multiple operation scheduling schemes of the sea-land collaboration-based multi-energy coupling low-carbon new energy system are calculated, an operation scheduling scheme that satisfies the constraints is screened out, a single-objective optimal operation scheduling scheme or a multi-objective optimal Pareto frontier is obtained using an optimization algorithm, and the sea-land collaboration-based multi-energy coupling low-carbon new energy system operates based on the single-objective optimal operation scheduling scheme or the multi-objective optimal Pareto frontier. The objective functions or constraints include:
a constraint of an ammonia synthesis process of an electro-catalytic ammonia production device, a formula of which is:

$$3 {n}_{{H}_{2} , {NH}_{3}}^{island} ( t ) + {n}_{{N}_{2}}^{island} ( t ) \to 2 {n}_{N {H}_{3} , prod}^{island} ( t ) ,$$

where, n_{H}_{₂}_{,NH}_{₃}^{island}(t) is a quantity of hydrogen consumed for ammonia production, n_{N}_{₂}^{island}(t) is a quantity of nitrogen consumed for ammonia production, and n_{NH}_{₃}_{,prod}^{island}(t) is a production output of ammonia;
a constraint of a methane synthesis process of a methane synthesis device, a formula of which is:

$$4 {n}_{{H}_{2} , {CH}_{4}}^{island} ( t ) + {n}_{C {O}_{2} , {CH}_{4}}^{island} ( t ) \to {n}_{C {H}_{4} , prod}^{island} ( t ) + 2 {H}_{2} O ,$$

where, n_{H}_{₂}_{,CH}_{₄}^{island}(t) is a quantity of hydrogen consumed for methane production, n_{CO}_{₂}_{,CH}_{₄}^{island}(t) is a quantity of carbon dioxide generated by methane combustion, and n_{CH}_{₄}_{,prod}^{island}(t) is a production output of methane;
a constraint of a methanol synthesis process of a methanol synthesis device, a formula of which is:

$$2 {n}_{{H}_{2} , {CH}_{3} OH}^{island} ( t ) + {n}_{C {O}_{2} , {CH}_{3} O H}^{island} ( t ) \to {n}_{C {H}_{3} OH , prod}^{island} ( t ) + {H}_{2} O ,$$

where, n_{H}_{₂}_{,CH}_{₃}_{OH}^{island}(t) is a quantity of hydrogen consumed for methanol production, island n_{CO}_{₂}_{,CH}_{₃}_{OH}^{island}(t) is a quantity of carbon dioxide consumed for methanol production, and n_{CH}_{₃}_{OH,prod}^{island}(t) is a production output of methanol;
energy consumption constraints for green fuel production, formulas of which are:

$$\begin{matrix}{P}_{{H}_{2} , prod}^{island} ( t ) = {f}_{{H}_{2} , p r o d}^{island} ( {n}_{{H}_{2} , prod}^{island} ( t ) ) , \\ {P}_{N {H}_{3} , prod}^{island} ( t ) = {f}_{N {H}_{3} , prod}^{island} ( {n}_{N {H}_{3} , prod}^{island} ( t ) ) , \\ {P}_{C {H}_{4} , prod}^{island} ( t ) = {f}_{C {H}_{4} , prod}^{island} ( {n}_{C {H}_{4} , prod}^{island} ( t ) ) , \\ {P}_{C {H}_{3} OH , prod}^{island} ( t ) = {f}_{C {H}_{3} O H , p r o d}^{island} ( {n}_{C {H}_{3} OH , prod}^{island} ( t ) ) ,\end{matrix}$$

where, f_{H}_{₂}_{,prod}^{island}, f_{NH}_{₃}_{,prod}^{island}, f_{CH}_{₄}_{,prod}^{island} and f_{CH}_{₃}_{OH,prod}^{island} are respectively energy consumption conversion relations in a green fuel production process; P_{H}_{₂}_{,prod}^{island}(t), P_{NH}_{₃}_{,prod}^{island}(t), P_{CH}_{₄}_{,prod}^{island}(t) and P_{CH}_{₃}_{OH,prod}^{island}(t) are respectively power consumption of hydrogen production, power consumption of ammonia production, power consumption of methane production and power consumption of methanol production at a time t; n_{H}_{₂}_{,prod}^{island}(t), n_{NH}_{₃}_{,prod}^{island}(t), n_{CH}_{₄}_{,prod}^{island}(t) and island n_{CH}_{₃}_{OH,prod}^{island}(t) are respectively production outputs of hydrogen, ammonia, methane and methanol at the time t;
energy consumption constraints of green fuel combustion, formulas of which are:

$$\begin{matrix}{P}_{N {H}_{3} , cb}^{island} ( t ) = {f}_{N {H}_{3} , c b}^{island} ( {n}_{N {H}_{3} , cb}^{island} ( t ) ) , \\ {P}_{C {H}_{4} , cb}^{island} ( t ) = {f}_{C {H}_{4} , cb}^{island} ( {n}_{C {H}_{4} , cb}^{island} ( t ) ) , \\ {P}_{C {H}_{3} OH , cb}^{island} ( t ) = {f}_{C {H}_{3} OH , cb}^{island} ( {n}_{C {H}_{3} OH , cb}^{island} ( t ) ) ,\end{matrix}$$

where, f_{NH}_{₃}_{,cb}^{island}, f_{CH}_{_{4,cb}}^{island} and f_{CH}_{₃}_{OH,cb}^{island} are respectively energy consumption conversion relations in a utilization process of island green fuels; P_{NH}_{₃}_{,cb}^{island}(t), P_{CH}_{₄}_{,cb}^{island}(t) and P_{CH}_{₃}_{OH,cb}^{island}(t) are respectively power of an ammonia power generator, power of a methane power generator and power of a methanol power generator at the time t; n_{NH}_{₃}_{,cb}^{island}(t), n_{CH}_{₄}_{,cb}^{island}(t) and n_{CH}_{₃}_{OH,cb}^{island}(t) are respectively a quantity of ammonia combusted on an island, a quantity of methane combusted on the island and a quantity of methanol combusted on the island;
a capacity constraint of an island hydrogen storage tank, a formula of which is:

$$\frac{( {n}_{{H}_{2} , charge}^{island} ( t ) - {n}_{{H}_{2} , discharge}^{island} ( t ) ) \times Δ t}{Capa {city}_{{H}_{2}}^{island}} = {SOC}_{{H}_{2}}^{island} ( t ) - S O {C}_{{H}_{2}}^{island} ( t - 1 ) ,$$

where, n_{H}_{₂}_{,charge}^{island}(t) and n_{H}_{₂}_{,discharge}^{island}(t) are respectively a quantity of hydrogen stored in the island hydrogen storage tank and a quantity of hydrogen discharged from the island hydrogen storage tank at the time t, Capacity_{H}_{₂}^{island} is a total capacity of the island hydrogen storage tank, and SOC_{H}_{₂}^{island}(t) and SOC_{H}_{₂}^{island}(t−1) are respectively a hydrogen storage capacity of the island hydrogen storage tank at the time t and a hydrogen storage capacity of the island hydrogen storage tank at a time (t−1);
a capacity constraint of a methane storage tank of a transport boat, a formula of which is:

$$\frac{( {n}_{C {H}_{4} , charge}^{b o a t} ( t ) - {n}_{C {H}_{4} , discharge}^{b o a t} ( t ) ) \times Δ t}{{Capacity}_{C {H}_{4}}^{b o a t}} = S O {C}_{C {H}_{4}}^{boat} ( t ) - S O {C}_{C {H}_{4}}^{b o a t} ( t - 1 ) ,$$

where, n_{CH}_{₄}_{,charge}^{boat}(t) and n_{CH}_{₄}_{,discharge}^{boat}(t) are respectively a quantity of methane stored in the methane storage tank of the transport boat and a quantity of methane discharged from the methane storage tank of the transport boat at the time t, Capacity_{CH}_{₄}^{boat} is a total capacity of the methane storage tank, and SOC_{CH}_{₄}^{boat}(t) and SOC_{CH}_{₄}^{boat}(t−1) are respectively a capacity of the methane storage tank of the transport boat at the time t and a capacity of the methane storage tank of the transport boat at the time (t−1);
a capacity constraint of a carbon dioxide storage tank of the transport boat, a formula of which is:

$$\frac{( {n}_{{CO}_{2} , charge}^{boat} ( t ) - {n}_{{CO}_{2} , discharge}^{boat} ( t ) ) \times Δ t}{{Capacity}_{{CO}_{2}}^{b o a t}} = S O {C}_{C {O}_{2}}^{boat} ( t ) - S O {C}_{C {O}_{2}}^{boat} ( t - 1 ) ,$$

where, n_{CO}_{₂}_{,charge}^{boat}(t) and n_{CO}_{₂}_{,discharge}^{boat}(t) are respectively a quantity of carbon dioxide stored in the carbon dioxide storage tank of the transport boat and a quantity of carbon dioxide discharged from the carbon dioxide storage tank of the transport boat at the time t, Capacity_{CO}_{₂}^{boat} is a total capacity of the carbon dioxide storage tank, and SOC_{CO}_{₂}^{boat}(t) and SOC_{CO}_{₂}^{boat}(t−1) are respectively a capacity of the carbon dioxide storage tank of the transport boat at the time t and a capacity of the carbon dioxide storage tank of the transport boat at the time (t−1);
an operating state constraint of the transport boat, a formula of which is:

$${I}_{c h a r g e} ( t ) + {I}_{d i s c h a r g e} ( t ) + {I}_{sailing} ( t ) + {I}_{m o o r i n g} ( t ) = 1 ,$$

where, I_{charge}(t) indicates that the boat is in a working medium charging state at the time t, I_{discharge}(t) indicates that the boat is in a working medium discharging state, I_{sailing}(t) indicates that the boat is in a sailing state, and I_{mooring}(t) indicates that the boat is in a mooring state.

In Step 5, the objective functions of the multiple operation scheduling schemes of the sea-land collaboration-based multi-energy coupling low-carbon new energy system include:
an operating cost objective function, a formula of which is:

$$cost ( t ) = ( {n}_{C {O}_{2} , emission}^{island} ( t ) + {n}_{C {O}_{2} , e m i s s i o n}^{land} ( t ) ) \times {Price}_{C {O}_{2}} + {n}_{coal , cb}^{land} ( t ) \times {Price}_{coal} ,$$

where, Price_{CO}_{₂} is a carbon sink price, Price_{coal} is a coal price, n_{CO}_{₂}_{,emission}^{island} is a quantity of carbon dioxide directly discharged by an island, n_{CO}_{₂}_{,emission}^{land}(t) is carbon dioxide emission of land, and n_{coal,cb}^{land}(t) is coal combustion of the land;
an operating revenue objective function, a formula of which is:

$$revenue ( t ) = ( {P}_{load}^{island} ( t ) + {P}_{d e m a n d}^{land} ( t ) ) \times {Price}_{electricity} ,$$

where, revenue(t) is the operating revenue objective function, and P_{load}^{island}(t) and P_{demand}^{island}(t) are respectively a power load of the island at a time t and a power demand of the island at the time t; Price_{electricity} is an electricity price.

In addition, the objective functions or constraints include a levelized cost of energy for evaluating economy, carbon emission per kilowatt-hour for evaluating environmental influences, and a load satisfaction rate for evaluating power supply reliability; and the optimization algorithm is a genetic algorithm or a particle swarm algorithm, or is solved using a linear programming solver. Wherein:
a formula of the levelized cost of energy is as follows:

$$LCOE = \frac{{\sum }_{i} {I}_{i} \times \frac{{{r}_{i} ( 1 + {r}_{i} )}^{{N}_{i}}}{{( 1 + {r}_{i} )}^{{N}_{i}} - 1} + ( O {M}_{i} + {V}_{i} )}{{\sum }_{t = 1}^{8760} {P}_{d e m and} ( t ) - {P}_{s h o r t a g e} ( t )} \qquad ( 3 )$$

where, the subscript i indicates different devices, I indicates an initial investment cost, OM indicates annual fixed operation and maintenance costs (including labor cost, management cost and the like), V indicates annual variable costs (mainly including coal cost), N indicates the life of devices, and r indicates a discount rate (8%);
a formula of the carbon emission per kilowatt-hour is as follows:

$$α = \frac{{m}_{coal} \times {θ}_{coal} + {m}_{N G} \times {θ}_{N G}}{{\sum }_{t = 1}^{8760} {P}_{d e m and} ( t ) - {P}_{s h o r t a g e} ( t )} \qquad ( 4 )$$

where, m_{coal} indicates coal consumption all year round, θ_{coal} indicates a carbon emission factor of coal, m_{NG} indicates natural gas consumption all year around, θ_{NG} indicates a carbon emission factor of natural gas, t indicates time, P_{demand} indicates a power demand, and P_{shortage} indicates a power shortage;
a formula of the load satisfaction rate is as follows:

$$Reliability = 1 - \frac{{\sum }_{t = 1}^{8760} {P}_{s h o r t a g e} ( t )}{{\sum }_{t = 1}^{8760} {P}_{d e m a n d} ( t )} \qquad ( 5 )$$

where, P_{demand} indicates a power demand, and P_{shortage} indicates a power shortage.

By multi-energy coupling, the power load satisfaction rate is greater than or equal to 99%, the light/wind curtailment rate is lower than 10%, and the carbon emission per kilowatt-hour of an energy supply system is decreased from 800 g/kWh to 300 g/kWh.

The sea-land resource collaborative and optimal scheduling method according to the disclosure may realize day-ahead scheduling, within-day scheduling and real-time scheduling according to the scheduling time.

According to day-ahead scheduling, an energy production and consumption plan is made one day ahead based on predicted data of a place where the sea-land collaborative energy system to optimize resource allocation so as to reduce the cost and maximize the revenue. In this stage, the sampling time is one hour, and in the scheduling process, not only the energy cost needs to be taken into account, but also the potential market return needs to be evaluated to make a risk-minimized production strategy, thus making a best balance between efficiency and cost of resources. The day-ahead scheduling process includes all decision variables.

Based on a day-ahead scheduling instruction, within-day scheduling reflects changes of the market and operation conditions within a shorter time, multiple updates are performed in the day to adjust a day-ahead plan to adapt to real-time changes, and the sampling time is generally 15 minutes. Within-day scheduling includes amendments to weather prediction, equipment performance changes or responses to sudden changes of the market demand. Within-day scheduling adjusts the power generation capacity and fuel use using a dynamic optimization model to maximize the cost effectiveness and guarantee the stability and reliability of system operation. In the decision-making process of within-day scheduling, the operating state of equipment with a low response speed (such as equipment for preparing ammonia, methane, methanol and other fuels) will not be adjusted.

Based on a within-day scheduling instruction, real-time scheduling adjusts the power balance between the island and the land based on real-time data and the instant system state and focuses on minute-level quick responses. During real-time scheduling, the system needs to quickly deal with emergencies (such as equipment faults or temporary load increases) and is kept stable by instant adjustment of operating parameters (such as the charge-discharge state of the energy storage device and emergent power generation scheduling). The challenge of real-time scheduling is to make an accurate decision within an extremely short time to guarantee both the economic benefits and operation safety.

By adopting such a multi-time scale scheduling method, the system may flexibly cope with various operation challenges to effectively manage energy flow and optimize the economy of the system and environmental influences.

The disclosure is described in detail above in conjunction with embodiments, but the above embodiments are merely preferred ones of the disclosure and should not be construed as limiting the implementation scope of the disclosure. All equivalent transformations and improvements made according to the application scope of the disclosure should also fall within the patent scope of the disclosure.

What is claimed is:

1. A sea-land collaboration-based energy system, comprising a low-carbon power generation unit, a green fuel synthesis unit and an energy storage device which are arranged on a sea and an island, a green fuel comprehensive utilization unit and a carbon capture device which are arranged on the island and/or on land, and a multi-energy flow coupling-based sea-land collaborative low-carbon intelligent control center arranged on the island or on land;
the low-carbon power generation unit generates zero-carbon and/or low-carbon power based on wind energy, light energy and nuclear energy on the sea and/or the island;
the green fuel synthesis unit produces hydrogen and ammonia using the zero-carbon and/or low-carbon power generated by the low-carbon power generation unit and produces methane and methanol using the hydrogen and carbon dioxide from the carbon capture device, and the ammonia, the methane and the methanol are used by the green fuel comprehensive utilization unit as green fuels;
the energy storage device performs charging using surplus power in a case where power is still surplus after the low-carbon power generation unit satisfies a power load, and compensates for a power shortage in a case where the low-carbon power generation fails to satisfy the power load;
the green fuel comprehensive utilization unit generates power preferentially using the green fuels in a case where the power load is still not satisfied after the energy storage device compensates for the power shortage, and generates power by combusting natural gas and/or coal in a case where the power load is still not satisfied after the green fuel comprehensive utilization unit generates power using the green fuels;
the carbon capture device is configured for capturing carbon dioxide produced by the green fuel comprehensive utilization unit and transporting the carbon dioxide into the green fuel synthesis unit;
the sea-land collaborative low-carbon intelligent control center calculates a zero-carbon and/or low-carbon power generation capacity according to acquired parameters of the wind energy, light energy and nuclear energy on the sea and/or the island, and controls operation strategies of the green fuel synthesis unit and the green fuel comprehensive utilization unit according to a power output of the energy storage device and the power load.

2. The sea-land collaboration-based energy system according to claim 1, wherein the green fuel synthesis unit comprises:
a seawater-electrolysis hydrogen production device driven by a wind-light volatile power supply, configured for producing hydrogen by seawater electrolysis;
an electro-catalytic ammonia production device, configured for synthesizing ammonia by hydrogen and nitrogen;
a methane synthesis device, configured for synthesizing methane using hydrogen and carbon dioxide; and
a methanol synthesis device, configured for synthesizing methanol using hydrogen and carbon dioxide;
a surface of an electrode used by the seawater-electrolysis hydrogen production device is covered with an alloy catalyst modified by mixing multiple elements, doped precious metal elements comprise one or more of nickel, ruthenium, cadmium, molybdenum and platinum, a doped metal loading capacity of an electrode catalyst is less than 0.5 mg/cm², a cathodic overpotential is less than or equal to 300 mV@1000 mA/cm², and an anodic overpotential is less than or equal to 600 mV@1000 mA/cm²; and an electro-catalytic material in the seawater-electrolysis hydrogen production device is a metal organic complex, a cathodic material is an Al—N₂ or Zr—N₂ cathodic material, and an electrolyte solution is one or more of ionic liquid, ionic liquid/organic solvent, and ionic liquid/organic solvent/water.

3. The sea-land collaboration-based energy system according to claim 1, further comprising a storage and transport unit, wherein the storage and transport unit comprises a transport boat commuting between the island and the land, and a green fuel receiving station, a detachable, assembleable and transportable floating high-pressure and low-temperature storage tank, an efficient and active heat-insulation system under an all-weather complex sea condition, and a safe and efficient green fuel injection system of an unstable platform, which are arranged on land;
the transport boat is configured for transporting the green fuels generated by the green fuel synthesis unit to the green fuel receiving station and transporting the carbon dioxide captured by the carbon capture device to the green fuel synthesis unit;
the green fuel receiving station is configured for receiving the green fuels from the transport boat;
the floating high-pressure and low-temperature storage tank is configured for storing the green fuels received by the green fuel receiving station;
the green fuel transfer system is configured for realizing offshore unattended docking transfer/filling of the green fuels to transport the green fuels to the green fuel comprehensive utilization unit or to an outside;
the heat-insulation system is configured for heat insulation of the transport boat, the green fuel receiving station, the floating high-pressure and low-temperature storage tank and the transfer system.

4. The sea-land collaboration-based energy system according to claim 3, wherein the green fuel receiving station comprises an ammonia storage device, a methane storage device and a methanol storage device.

5. The sea-land collaboration-based energy system according to claim 4, wherein the energy storage device comprises one or a combination of electrochemical energy storage and thermal energy storage; and the green fuel comprehensive utilization unit comprises a coal-fired power generation device, a natural gas methane power generation device, a methanol power generation device, an ammonia released energy power generation device, the natural gas methane power generation device is connected to the methane storage device of the green fuel receiving station, the methanol power generation device is connected to the methanol storage device of the green fuel receiving station, and the ammonia released energy power generation device is connected to the ammonia storage device of the green fuel receiving station.

6. The sea-land collaboration-based energy system according to claim 1, wherein the carbon capture device captures carbon dioxide in smoke generated by the green fuel comprehensive utilization unit using an absorption solution through a chemical absorption method.

7. A sea-land resource collaborative and optimal scheduling method based on the sea-land collaboration-based energy system according to claim 1, comprising the following steps:
step 1, acquiring natural resource parameters of wind energy and light energy at multiple time scales, and calculating a power output of the low-carbon power generation unit according to the natural resource parameters and a configurated capacity of the low-carbon power generation unit;
step 2, inputting a local power load, and calculating a power surplus based on the power output of the low-carbon power generation unit and the power load; if the power surplus is greater than 0, producing green fuels using the green fuel synthesis unit to consume surplus power and/or storing surplus power using the energy storage device, and updating an energy storage capacity of the energy storage device; or, if the power surplus is less than or equal to 0, compensating for a power shortage using the energy storage device and generating green fuels using the green fuel synthesis unit, and updating the energy storage capacity of the energy storage device, wherein the produced green fuels comprise ammonia, methane and methanol, an operation strategy of the green fuel synthesis unit (i.e., output proportions of ammonia, methane and methanol) is set, and total carbon consumption is calculated;
step 3, redetermining whether the power shortage still exits; if the power shortage still exits, making an operation strategy of the green fuel comprehensive utilization unit, wherein the operation strategy of the green fuel comprehensive utilization unit is set for maximizing a power output of the green fuels and minimizing a power output of carbon fuels to make up for the power shortage;
step 4, calculating a carbon output of the green fuel comprehensive utilization unit based on the operation strategy made in step 3, and comparing the carbon output of the green fuel comprehensive utilization unit with the total carbon consumption calculated in step 2; if the carbon output is less than or equal to the total carbon consumption, returning to step 3, adjusting the operation strategy of the green fuel comprehensive utilization unit, increasing the power output of the carbon fuels, and decreasing the power output of the green fuels; or, if the carbon output is greater than the total carbon consumption, calculating total carbon emission; if the total carbon emission does not satisfy emission and design requirements, returning to step 2, adjusting the operation strategy of the green fuel synthesis unit, increasing the output proportions of methane and methanol, and deceasing the output proportion of ammonia; when the carbon output is less than or equal to the total carbon consumption and the total carbon emission satisfies the emission and design requirements, taking one of the adjusted operation strategy of the green fuel synthesis unit and the adjusted operation strategy of the green fuel comprehensive utilization unit as an operation scheduling scheme of the sea-land collaboration-based multi-energy coupling low-carbon new energy system; and
step 5, repeating steps 1-4 to form multiple operation scheduling schemes of the sea-land collaboration-based multi-energy coupling low-carbon new energy system, screening out an operation scheduling scheme that satisfies objective functions and constraints, obtaining a single-objective optimal operation scheduling scheme or a multi-objective optimal Pareto frontier using an optimization algorithm, and operating the sea-land collaboration-based multi-energy coupling low-carbon new energy system based on the single-objective optimal operation scheduling scheme or the multi-objective optimal Pareto frontier.

8. The sea-land resource collaborative and optimal scheduling method according to claim 7, wherein the operation scheduling schemes formed in Step 4 satisfy the following balances:
island hydrogen satisfies the following balance:
$${n}_{{H}_{2} , prod}^{island} ( t ) - {n}_{{H}_{2} , {NH}_{3}}^{island} ( t ) - {n}_{{H}_{2} , {CH}_{4}}^{island} ( t ) - {n}_{{H}_{2} , {CH}_{3} OH}^{island} ( t ) - {n}_{{H}_{2} , charge}^{island} ( t ) + {n}_{{H}_{2} , discharge}^{island} ( t ) = 0$$
wherein, n_{H}_{₂}_{,prod}^{island}(t) is a production output of hydrogen, N_{H}_{₂}_{,NH}_{₃}^{island}(t) is a quantity of hydrogen consumed for ammonia production, n_{H}_{₂}_{,CH}_{₄}^{island}(t) is a quantity of hydrogen consumed for methane production, n_{H}_{₂}_{,CH}_{₃}_{OH}^{island}(t) is a quantity of hydrogen consumed for methanol production, n_{H}_{₂}_{,charge}^{island}(t) is a quantity of hydrogen stored in an island hydrogen storage device, and n_{H}_{₂}_{,discharge}^{island}(t) is a quantity of hydrogen discharged from the island hydrogen storage device;
ammonia satisfies the following balance:
$${n}_{{NH}_{3} , prod}^{island} ( t ) - {n}_{{NH}_{3} , charge}^{boat} ( t ) - {n}_{{NH}_{3} , cb}^{island} ( t ) - {n}_{{NH}_{3} , charge}^{island} ( t ) + {n}_{{NH}_{3} , discharge}^{island} ( t ) = 0$$
wherein, N_{NH}_{₃}_{,prod}^{island}(t) is a production output of ammonia, n_{NH}_{₃}_{,charge}^{boat}(t) is a quantity of ammonia loaded onto a boat, n_{NH}_{₃}_{,cb}^{island}(t) is a quantity of ammonia combusted on an island, n_{NH}_{₃}_{,charge}^{island}(t) is a quantity of ammonia stored in an island ammonia storage device, and n_{NH}_{₃}_{,discharge}^{island}(t) is a quantity of ammonia discharged from the island ammonia storage device;
methane satisfies the following balance:
$${n}_{{CH}_{4} , prod}^{island} ( t ) - {n}_{{CH}_{4} , charge}^{boat} ( t ) - {n}_{{CH}_{4} , cb}^{island} ( t ) - {n}_{{CH}_{4} , charge}^{island} ( t ) + {n}_{{CH}_{4} , discharge}^{island} ( t ) = 0$$
wherein, n_{CH}_{₄}_{,prod}^{island}(t) is a production output of methane, n_{CH}_{₄}_{,charge}^{island}(t) is a quantity of methane loaded onto the boat, n_{CH}_{₄}_{,cb}^{island}(t) is a quantity of methane combusted on the island, n_{CH}_{₄}_{,charge}^{island}(t) is a quantity of methane stored in an island methane storage device, and n_{CH}_{₄}_{,discharge}^{island}(t) is a quantity of methane discharged from the island methane storage device;
methanol satisfies the following balance:
$${n}_{{CH}_{3} OH , prod}^{island} ( t ) - {n}_{{CH}_{3} OH , charge}^{boat} ( t ) - {n}_{{CH}_{3} OH , cb}^{island} ( t ) - {n}_{{CH}_{3} OH , charge}^{island} ( t ) + {n}_{{CH}_{3} OH , discharge}^{island} ( t ) = 0$$
wherein, n_{CH}_{₃}_{OH,prod}^{island}(t) is a production output of methanol, n_{CH}_{₃}_{OH,charge}^{boat}(t) is a quantity of methanol loaded onto the boat, n_{CH}_{₃}_{OH,cb}^{island}(t) is a quantity of methanol combusted on the island, n_{CH}_{₃}_{OH,charge}^{island}(t) is a quantity of methanol stored in an island methanol storage device, and n_{CH}_{₃}_{OH,discharge}^{island}(t) is a quantity of methanol discharged from the island methanol storage device;
carbon dioxide satisfies the following balance:
$${n}_{{CO}_{2} , {CH}_{4}}^{island} ( t ) + {n}_{{CO}_{2} , {CH}_{3} OH}^{island} ( t ) + {n}_{{CO}_{2} , coal}^{island} ( t ) - {n}_{{CO}_{2} , capture}^{island} ( t ) + {n}_{{CO}_{2} , discharge}^{boat} ( t ) - {n}_{{CO}_{2} , emission}^{island} ( t ) = 0$$
wherein, n_{CO}_{₂}_{,CH}_{₄}^{island}(t) is a quantity of carbon dioxide produced by methane combustion, n_{CO}_{₂}_{,CH}_{₃}_{,OH}^{island}(t) is a quantity of carbon dioxide produced by methanol combustion, n_{CO}_{₂}_{,coal}^{island}(t) is a quantity of carbon dioxide produced by coal combustion, n_{CO}_{₂}_{,capture}^{island}(t) is a quantity of carbon dioxide captured by an island carbon capture facility, n_{CO}_{₂}_{,discharge}^{boat}(t) is a quantity of carbon dioxide discharged by the boat, and n_{CO}_{₂}_{,emission}^{island}(t) is a quantity of carbon dioxide directly discharged by the island;
power satisfies the following balance:
$${P}_{wind}^{island} ( t ) + {P}_{solar}^{island} ( t ) + {P}_{{NH}_{3} , cb}^{island} ( t ) + {P}_{{CH}_{4} , cb}^{island} ( t ) + {P}_{{CH}_{3} OH , cb}^{island} ( t ) - {P}_{{H}_{2} , prod}^{island} ( t ) - {P}_{{NH}_{3} , prod}^{island} ( t ) - {P}_{{CH}_{4} , prod}^{island} ( t ) - {P}_{{CH}_{3} OH , prod}^{island} ( t ) = {P}_{load}^{island} ( t )$$
wherein, P_{wind}^{island}(t), Pₛₒₗₐᵣ^{island}(t), P_{NH}_{₃}_{,cb}^{island}(t), P_{CH}_{₄}_{,cb}^{island}(t) and P_{CH}_{₃}_{OH,cb}^{island}(t) are respectively power of a wind power generator, power of a solar power generator, power of an ammonia power generator, power of a methane power generator and power of a methanol power generator; P_{H}_{₂}_{,prod}^{island}(t), P_{NH}_{₃}_{,prod}^{island}(t), P_{CH}_{₄}_{,prod}^{island}(t) and P_{CH}_{₃}_{OH,prod}^{island}(t) are respectively power consumption of hydrogen production, power consumption of ammonia production, power consumption of methane production and power consumption of methanol production; P_{load}^{island}(t) is a real-time power load of the island.

9. The sea-land resource collaborative and optimal scheduling method according to claim 7, wherein the constraints in step 5 comprise:
a constraint of an ammonia synthesis process of an electro-catalytic ammonia production device, a formula of which is:
$$3 {n}_{{H}_{2} , {NH}_{3}}^{island} ( t ) + {n}_{{N}_{2}}^{island} ( t ) \to 2 {n}_{{NH}_{3} , prod}^{island} ( t ) ,$$
wherein, n_{H}_{₂}_{,NH}_{₃}^{island}(t) is a quantity of hydrogen consumed for ammonia production, n_{N}_{₂}^{island}(t) is a quantity of nitrogen consumed for ammonia production, and n_{NH}_{₃}_{,prod}^{island}(t) is a production output of ammonia;
a constraint of a methane synthesis process of a methane synthesis device, a formula of which is:
$$4 {n}_{{H}_{2} , {CH}_{4}}^{island} ( t ) + {n}_{{CO}_{2} , {CH}_{4}}^{island} ( t ) \to {n}_{{CH}_{4} , prod}^{island} ( t ) + 2 {H}_{2} O ,$$
wherein, n_{H}_{₂}_{,CH}_{₄}^{island}(t) is a quantity of hydrogen consumed for methane production, n_{CO}_{₂}_{,CH}_{₄}^{island}(t) is a quantity of carbon dioxide generated by methane combustion, and n_{CH}_{₄}_{,prod}^{island}(t) is a production output of methane;
a constraint of a methanol synthesis process of a methanol synthesis device, a formula of which is:
$$2 {n}_{{H}_{2} , {CH}_{3} OH}^{island} ( t ) + {n}_{{CO}_{2} , {CH}_{3} OH}^{island} ( t ) \to {n}_{{CH}_{3} OH , prod}^{island} ( t ) + {H}_{2} O ,$$
wherein, n_{H}_{₂}_{,CH}_{₃}_{OH}^{island}(t) is a quantity of hydrogen consumed for methanol production, n_{CO}_{₂}_{,CH}_{₃}_{OH}^{island}(t) is a quantity of carbon dioxide consumed for methanol production, and n_{CH}_{₃}_{OH,prod}^{island}(t) is a production output of methanol;
energy consumption constraints for green fuel production, formulas of which are:
$${P}_{{H}_{2} , prod}^{island} ( t ) = {f}_{{H}_{2} , prod}^{island} ( {n}_{{H}_{2} , prod}^{island} ( t ) ) , \\ {P}_{{NH}_{3} , prod}^{island} ( t ) = {f}_{{NH}_{3} , prod}^{island} ( {n}_{{NH}_{3} , prod}^{island} ( t ) ) , \\ {P}_{{CH}_{4} , prod}^{island} ( t ) = {f}_{{CH}_{4} , prod}^{island} ( {n}_{{CH}_{4} , prod}^{island} ( t ) ) , \\ {P}_{{CH}_{3} OH , prod}^{island} ( t ) = {f}_{{CH}_{3} OH , prod}^{island} ( {n}_{{CH}_{3} OH , prod}^{island} ( t ) ) ,$$
wherein, f_{H}_{₂}_{,prod}^{island}, f_{NH}_{₃}_{,prod}^{island}, f_{CH}_{₄}_{,prod}^{island} and f_{CH}_{₃}_{OH,prod}^{island} are respectively energy consumption conversion relations in a green fuel production process; P_{H}_{₂}_{,prod}^{island}(t), P_{NH}_{₃}_{,prod}^{island}(t), P_{CH}_{₄}_{,prod}^{island}(t) and P_{CH}_{₃}_{OH,prod}^{island}(t) are respectively power consumption of hydrogen production, power consumption of ammonia production, power consumption of methane production and power consumption of methanol production at a time t; n_{H}_{₂}_{,prod}^{island}(t), n_{NH}_{₃}_{,prod}^{island}(t), n_{CH}_{₄}_{,prod}^{island}(t) and island n_{CH}_{₃}_{OH,prod}^{island}(t) are respectively production outputs of hydrogen, ammonia, methane and methanol at the time t;
energy consumption constraints of green fuel combustion, formulas of which are:
$${P}_{{NH}_{3} , cb}^{island} ( T ) = {f}_{{NH}_{3} , cb}^{island} ( {n}_{{NH}_{3} , cb}^{island} ( T ) ) , \\ {P}_{{CH}_{4} , cb}^{island} ( T ) = {f}_{{CH}_{4} , cb}^{island} ( {n}_{{CH}_{4} , cb}^{island} ( T ) ) , \\ {P}_{{CH}_{3} OH , cb}^{island} ( T ) = {f}_{{CH}_{3} OH}^{island} ( {n}_{{CH}_{3} OH , cb}^{island} ( T ) ) ,$$
wherein, f_{NH}_{₃}_{,cb}^{island}, f_{CH}_{_{4,cb}}^{island} and f_{CH}_{₃}_{OH,cb}^{island} are respectively energy consumption conversion relations in a utilization process of island green fuels; P_{NH}_{₃}_{,cb}^{island}(t), P_{CH}_{₄}_{,cb}^{island}(t) and P_{CH}_{₃}_{OH,cb}^{island}(t) are respectively power of an ammonia power generator, power of a methane power generator and power of a methanol power generator at the time t; n_{NH}_{₃}_{,cb}^{island}(t), n_{CH}_{₄}_{,cb}^{island}(t) and n_{CH}_{₃}_{OH,cb}^{island}(t) are respectively a quantity of ammonia combusted on an island, a quantity of methane combusted on the island and a quantity of methanol combusted on the island;
a capacity constraint of an island hydrogen storage tank, a formula of which is:
$$\frac{( {n}_{{H}_{2} , charge}^{island} ( t ) - {n}_{{H}_{2} , discharge}^{island} ( t ) ) \times Δ t}{{Capacity}_{{H}_{2}}^{island}} = {SOC}_{{H}_{2}}^{island} ( t ) - {SOC}_{{H}_{2}}^{island} ( t - 1 ) ,$$
wherein, n_{H}_{₂}_{,charge}^{island}(t) and n_{H}_{₂}_{,discharge}^{island}(t) are respectively a quantity of hydrogen stored in the island hydrogen storage tank and a quantity of hydrogen discharged from the island hydrogen storage tank at the time t, Capacity_{H}_{₂}^{island} is a total capacity of the island hydrogen storage tank, and SOC_{H}_{₂}^{island}(t) and SOC_{H}_{₂}^{island}(t−1) are respectively a hydrogen storage capacity of the island hydrogen storage tank at the time t and a hydrogen storage capacity of the island hydrogen storage tank at a time (t−1);
a capacity constraint of a methane storage tank of a transport boat, a formula of which is:
$$\frac{( {n}_{{CH}_{4} , charge}^{boat} ( t ) - {n}_{{CH}_{4} , discharge}^{boat} ( t ) ) \times Δ t}{{Capacity}_{{CH}_{4}}^{boat}} = {SOC}_{{CH}_{4}}^{boat} ( t ) - {SOC}_{{CH}_{4}}^{boat} ( t - 1 ) ,$$
where, n_{CH}_{₄}_{,charge}^{boat}(t) and n_{CH}_{₄}_{,discharge}^{boat}(t) are respectively a quantity of methane stored in the methane storage tank of the transport boat and a quantity of methane discharged from the methane storage tank of the transport boat at the time t, Capacity_{CH}_{₄}^{boat} is a total capacity of the methane storage tank, and SOC_{CH}_{₄}^{boat}(t) and SOC_{CH}_{₄}^{boat}(t−1) are respectively a capacity of the methane storage tank of the transport boat at the time t and a capacity of the methane storage tank of the transport boat at the time (t−1);
a capacity constraint of a carbon dioxide storage tank of the transport boat, a formula of which is:
$$\frac{( {n}_{{CO}_{2} , charge}^{boat} ( t ) - {n}_{{CO}_{2} , discharge}^{boat} ( t ) ) \times Δ t}{{Capacity}_{{CO}_{2}}^{boat}} = {SOC}_{{CO}_{2}}^{boat} ( t ) - {SOC}_{{CO}_{2}}^{boat} ( t - 1 ) ,$$
where, n_{CO}_{₂}_{,charge}^{boat}(t) and n_{CO}_{₂}_{,discharge}^{boat}(t) are respectively a quantity of carbon dioxide stored in the carbon dioxide storage tank of the transport boat and a quantity of carbon dioxide discharged from the carbon dioxide storage tank of the transport boat at the time t, Capacity_{CO}_{₂}^{boat} is a total capacity of the carbon dioxide storage tank, and SOC_{CO}_{₂}^{boat}(t) and SOC_{CO}_{₂}^{boat}(t−1) are respectively a capacity of the carbon dioxide storage tank of the transport boat at the time t and a capacity of the carbon dioxide storage tank of the transport boat at the time (t−1);
an operating state constraint of the transport boat, a formula of which is:
$${I}_{charge} ( t ) + {I}_{discharge} ( t ) + {I}_{sailing} ( t ) + {I}_{mooring} ( t ) = 1$$
wherein, I_{charge}(t) indicates that the boat is in a working medium charging state at the time t, I_{discharge}(t) indicates that the boat is in a working medium discharging state, I_{sailing}(t) indicates that the boat is in a sailing state, and I_{mooring}(t) indicates that the boat is in a mooring state.

10. The sea-land resource collaborative and optimal scheduling method according to claim 7, wherein the objective functions in step 5 comprise:
an operating cost objective function, a formula of which is:
$$cost ( t ) = ( {n}_{{CO}_{2} , emission}^{island} ( t ) + {n}_{{CO}_{2} , emission}^{land} ( t ) ) \times {Price}_{{CO}_{2}} + {n}_{coal , cb}^{land} ( t ) \times {Price}_{coal}$$
wherein, Price_{CO}_{₂} is a carbon sink price, Price_{coal} is a coal price, n_{CO}_{₂}_{,emission}^{island} is a quantity of carbon dioxide directly discharged by an island, n_{CO}_{₂}_{,emission}^{land}(t) is carbon dioxide emission of land, and n_{coal,cb}^{land}(t) is coal combustion of the land;
an operating revenue objective function, a formula of which is:
$$revenue ( t ) = ( {P}_{load}^{island} ( t ) + {P}_{demand}^{land} ( t ) ) \times {Price}_{electricity}$$
wherein, revenue(t) is the operating revenue objective function, and P_{load}^{island}(t) and P_{demand}^{land}(t) are respectively a power load of the island at a time t and a power demand of the island at the time t; Price_{electricity} is an electricity price.

11. The sea-land resource collaborative and optimal scheduling method according to claim 7, wherein in step 4, a surplus of the green fuels is calculated according to the operation strategy of the green fuel synthesis unit and the operation strategy of the green fuel comprehensive utilization unit, and the calculated surplus is taken as a quantity of green fuels transported to an outside.
